(19) United States
(12) Patent Application Publication
(10) Pub. No.: US 2026/0363964 A1
(43) Pub. Date: Nov. 19, 2026

(54) METHOD FOR PREPARING ENDODONTIC FILLING MATERIAL

(71) Applicant: Ming-Kuan Biomedical Technology Corporation, Hsinchu County (TW)

(72) Inventors: Glemarie Coloma HERMOSA, Hsinchu County (TW); An-Cheng SUN, Hsinchu County (TW); Jing-Wei HONG, Hsinchu County (TW); Kuan-Wei LU, Hsinchu County (TW); Jayaraman ARCHANA, Taoyuan City (TW)

(21) Appl. No.: 19/209,052

(22) Filed: May 15, 2025

(51) Int. Cl. A61K 6/54; A61K 6/824; A61K 6/853; C04B 35/622

(52) U.S. Cl. CPC A61K 6/54; A61K 6/824; A61K 6/853; C04B 35/622

(57) ABSTRACT

A method for preparing an endodontic filling material includes the steps of: a) dispersing tricalcium silicate and tantalum pentoxide in a dispersing agent so as to obtain a first mixture; b) introducing the first mixture into an autoclave, and sealing and heating the autoclave, so that the first mixture is subjected to a one-pot treatment to obtain a second mixture which includes the dispersing agent and an endodontic filling material containing the tricalcium silicate and the tantalum pentoxide coated onto the tricalcium silicate; and c) subjecting the second mixture to filtration, so as to remove the dispersing agent therefrom, thereby obtaining the endodontic filling material.

### FIELD

The disclosure relates to a method for preparing an endodontic filling material. The disclosure also relates to an endodontic filling material prepared by the method and an endodontic filling product including the same.

### BACKGROUND

Pulpitis is a public health problem prevalent around the world, and is an inflammatory process of a dental pulp, which contains blood vessels, nerves, connective tissues, and so forth. Pulpitis may be caused by some reasons, such as untreated caries or dental trauma, leading to bacterial invasion from an enamel through a dentin to a pulp of a tooth.

An endodontic filling material for treatment of pulpitis normally has characteristics of being easily introduced into a root canal where an infected tooth pulp resides, being bacteriostatic, being biocompatible, being radio-opaque, having a good flow property, and being capable of hardening into a cement upon contact of a physiological environment. Examples of such endodontic filling material may include calcium hydroxide, resin modified glass ionomer cement, mineral trioxide aggregate (MTA), etc. Among the candidates suitable for use as the endodontic filling material, the MTA, which is a mixture normally containing tricalcium silicate and dicalcium silicate, tricalcium aluminate, tetracalcium aluminoferrite, and gypsum, has been reported to be a promising one in terms of biocompatibility and hydrophilicity.

However, a commercially available MTA product may show some disadvantages such as a potential of discoloration due to containing bismuth oxide, high cost, and having relatively large particle sizes (in micrometer scale) as well as a wide particle size distribution, both of which adversely affect the flow stability thereof and make the product require a long setting time. As a result, those skilled in the art strive to find a way to solve the foregoing problems.

### SUMMARY

Accordingly, an object of the present disclosure is to provide a method for preparing an endodontic filling material that can alleviate at least one of the drawbacks of the prior art.

The method includes the steps of:
a) dispersing tricalcium silicate and tantalum pentoxide in a dispersing agent so as to obtain a first mixture;
b) introducing the first mixture into an autoclave, and sealing and heating the autoclave, so that the first mixture is subjected to a one-pot treatment to obtain a second mixture which includes the dispersing agent and an endodontic filling material containing the tricalcium silicate and the tantalum pentoxide coated onto the tricalcium silicate; and
c) subjecting the second mixture to filtration, so as to remove the dispersing agent therefrom, thereby obtaining the endodontic filling material.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Other features and advantages of the disclosure will become apparent in the following detailed description of the embodiment(s) with reference to the accompanying drawings. It is noted that various features may not be drawn to scale.

FIG. 1 is a dynamic light scattering (DLS) graph illustrating particle size distribution of an endodontic filling material of Example 5.

FIG. 2 is an image, which is obtained by scanning electron microscope-energy dispersive X-ray spectroscopy (SEM-EDS), and which illustrates morphology and elements of the endodontic filling material of Example 5.

FIG. 3 is X-ray diffraction (XRD) patterns of a tricalcium silicate powder and a tantalum pentoxide powder of General Experimental Materials.

FIG. 4 is X-ray diffraction (XRD) patterns of the endodontic filling material of Example 5, an endodontic filling material of Example 8, and an endodontic filling material of Example 11, respectively.

FIG. 5 is X-ray images illustrating radiopacity effect of samples of Examples 1 to 12, respectively.

FIG. 6 is X-ray images illustrating radiopacity effect of samples of Comparative Examples 1 and 2, respectively.

### DETAILED DESCRIPTION

It is to be understood that, if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to,” and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for preparing an endodontic filling material, which includes the following steps a) to c).

First, in step a), tricalcium silicate (3CaO·SiO₂; “C₃S” in abbreviation) (particle size: approximately ranging from 300 nm to 700 nm) and tantalum pentoxide (Ta₂O₅; also known as tantalum(V) oxide) (particle size: approximately ranging from 300 nm to 800 nm) are dispersed in a dispersing agent so as to obtain a first mixture. In certain embodiments, a weight ratio of the tricalcium silicate to the tantalum pentoxide may range from 1.8:1 to 3:1. In an exemplary embodiment, a weight ratio of the tricalcium silicate to the tantalum pentoxide is 1.8:1. In another exemplary embodiment, a weight ratio of the tricalcium silicate to the tantalum pentoxide is 3:1. Each of the tricalcium silicate and the tantalum pentoxide may be in powder form. Additionally, the dispersing agent may be ethanol, and amount of the dispersing agent is not particularly limited as long as the amount thereof is appropriate for the method of the present disclosure.

According to the present disclosure, the tricalcium silicate has bioactivity and can provide an antimicrobial protection for a tooth, and the main function thereof is to seal a root canal system of the tooth. The tantalum pentoxide is greatly considered in orthopedic application, and is used to serve as a radiopacifying agent.

To be specific, the tricalcium silicate and the tantalum pentoxide may be added simultaneously (together) into the dispersing agent, and dispersing of the tricalcium silicate and the tantalum pentoxide (i.e., step a)) may be carried out by stirring, which enhances homogeneity of the first mixture. In some embodiments, the stirring may be conducted at room temperature for 1 hour.

In step b), the first mixture is introduced into an autoclave, and then the autoclave is sealed and heated, so that the first mixture is subjected to a one-pot treatment to obtain a second mixture which includes the dispersing agent and an endodontic filling material containing the tricalcium silicate and the tantalum pentoxide coated onto the tricalcium silicate. In certain embodiments, the one-pot treatment may be conducted at a temperature ranging from 120 °C to 220 °C (e.g., 140 °C to 200 °C, 160 °C to 200 °C) and a pressure ranging from 1 MPa to 1.2 MPa. In certain embodiments, the one-pot treatment may be conducted for a time period ranging from 2 hours to 10 hours (e.g., 4 hours to 10 hours). In certain embodiments, the autoclave may be heated using an oven.

In some embodiments, the endodontic filling material may have a particle size ranging from 200 nm to 1000 nm. In still some embodiments, the endodontic filling material may have a particle size ranging from 300 nm to 600 nm. In yet some embodiments the endodontic filling material may have a particle size ranging from 300 nm to 500 nm.

In an exemplary embodiment, the procedures for conducting the one-pot treatment is to, initially, introduce the first mixture into the autoclave, and then the autoclave containing the first mixture is sealed and placed into the oven, followed by heating the autoclave with the oven, so as to allow the first mixture in the autoclave to be heated to a predetermined temperature (e.g., 160 °C) at a heating rate of 18 °C/min (which causes the pressure of the autoclave to raise to a predetermined degree, e.g., 1.2 MPa). The first mixture in the autoclave is then kept at such predetermined temperature and such predetermined pressure for a time period (e.g., 6 hours), by which the tantalum pentoxide is coated onto the tricalcium silicate to form the endodontic filling material, so as to obtain the second mixture containing the endodontic filling material and the dispersing agent. Specifically, the endodontic filling material contained in the second mixture is formed from coating surfaces of the tricalcium silicate with the tantalum pentoxide. In some embodiments, the endodontic filling material may further contain dicalcium silicate which is coated with the tantalum pentoxide. In still some embodiments, based on 100 wt. % of a total amount of the tricalcium silicate and the dicalcium silicate, the tricalcium silicate is present in an amount of 90 wt. %, and the dicalcium silicate is present in an amount of 10 wt. %.

Finally, in step c), the second mixture is subjected to filtration, so as to remove the dispersing agent therefrom, thereby obtaining the endodontic filling material. For the filtration of the second mixture, a vacuum pump system including a filter paper may be used, and by pouring the second mixture onto the filter paper while simultaneously applying vacuum, the dispersing agent in the second mixture is filtered out from the filter paper and the endodontic filling material is left thereon. In certain embodiments, the filtration may be conducted for a time period ranging from 40 seconds to 90 seconds (e.g., 60 seconds to 90 seconds). Thereafter, the endodontic filling material left on the filter paper may be transferred to a glass plate. In certain embodiments, after step c), the endodontic filling material may be dried at 80 °C for 24 hours, so as to remove moisture remaining therein.

The present disclosure also provides the aforesaid endodontic filling material, which includes a plurality of particles containing tricalcium silicate nanoparticles and tantalum pentoxide nanoparticles coated on the tricalcium silicate nanoparticles. In certain embodiments, the endodontic filling material may be in powder form. In some embodiments, the plurality of particles of the endodontic filling material may have particle sizes ranging from 200 nm to 1000 nm. In still some embodiments, the plurality of particles of the endodontic filling material may have the particle sizes ranging from 300 nm to 600 nm. In yet some embodiments, the plurality of particles of the endodontic filling material may have the particle sizes ranging from 300 nm to 500 nm.

By virtue of the one-pot treatment, the procedures in the method for preparing the endodontic filling material are greatly simplified, and time consumption thereof can be reduced, thereby lowering the cost of the endodontic filling material. In addition, the endodontic filling material thus obtained has a nano-scale particle size, which enables the endodontic filling material to have good flow stability, and reduces setting time thereof.

The present disclosure also provides an endodontic filling material, which is prepared by the above-mentioned method.

Moreover, the present disclosure further provides an endodontic filling product, which contains a sealing agent and the aforesaid endodontic filling material. A polymer that is biocompatible may be used as the sealing agent. In certain embodiments, the sealing agent may be a polyethylene glycol (PEG), a polyvinyl alcohol (PVA), or a polylactic acid (PLA). In certain embodiments, the sealing agent may be present in an amount ranging from 20 wt. % to 40 wt. % based on 100 wt. % of the endodontic filling product. In some embodiments, the endodontic filling product may be in an injectable dosage form.

The present disclosure will be further described by way of the following examples. However, it should be understood that the following examples are intended solely for the purpose of illustration and should not be construed as limiting the present disclosure in practice

### EXAMPLES

### General Experimental Materials

1. Tricalcium silicate (C₃S) powder was purchased from Ming-Kuan Biomedical Technology Co., Ltd. and produced by the method as described in Taiwanese Invention Patent Certificate No. TW I771148, and product number thereof was MKC3S0070.
2. Tantalum pentoxide (Ta₂O₅) powder was purchased from Elecmat Technology Co., Ltd., and product number thereof was X11303060009.

### EXAMPLES

### Preparation of Endodontic Filling Material According to the Present Disclosure

### Example 1 (E1)

First, 3 g of the tricalcium silicate powder (peak particle size: 541.3 nm) and 1 g of the tantalum pentoxide powder (peak particle size: approximately 508.7 nm) were simultaneously added into 250 mL of ethanol (serving as a dispersing agent), followed by stirring for dispersing the tricalcium silicate powder and the tantalum pentoxide powder in the ethanol at room temperature for 1 hour, so as to obtain a first mixture. Next, the first mixture was introduced into an autoclave (KINGMALL INSTRUMENTS CO., LTD.; Model: 250 ml), followed by sealing the autoclave. Thereafter, the autoclave that is sealed was placed in an oven, and then heated using the oven to a temperature of 140 °C at a heating rate of 18 °C/min. During the heating process, the pressure in the autoclave was raised to 1.2 MPa. The first mixture was kept at such temperature (i.e., 140 °C) and pressure (i.e., 1.2 MPa) for 2 hours, which allows the first mixture to undergo a one-pot treatment, so that the tantalum pentoxide powder was coated onto the tricalcium silicate powder to form an endodontic filling material of E1, thereby obtaining a second mixture containing the endodontic filling material of E1 and the ethanol. Subsequently, the second mixture was subjected to filtration using a vacuum pump system having a filter paper (Advantec; Model: 5C/90 mm; pore size thereof: 1 um) for a time period within 1 minute, so as to remove the ethanol from the second mixture, thereby obtaining the endodontic filling material of E1. After that, the endodontic filling material of E1 was dried at 80 °C for 24 hours, so as so remove moisture remaining therein, thereby obtaining the endodontic filling material of E1 (peak particle size: 414.5 nm) in powder form.

### Example 2 (E2)

The procedures for preparing the endodontic filling material of E2 (presented in powder form) were similar to those of E1, except that the one-pot treatment was conducted for 6 hours, that is, the first mixture was kept at 140 °C and 1.2 MPa for 6 hours.

### Example 3 (E3)

The procedures for preparing the endodontic filling material of E3 (presented in powder form) were similar to those of E1, except that the one-pot treatment was conducted for 10 hours, that is, the first mixture was kept at 140 °C and 1.2 MPa for 10 hours.

### Example 4 (E4)

The procedures for preparing the endodontic filling material of E4 (presented in powder form) were similar to those of E1, except that the one-pot treatment was conducted at 160 °C, that is, the first mixture was kept at 160 °C and 1.2 MPa for 2 hours.

### Example 5 (E5)

The procedures for preparing the endodontic filling material of E5 (presented in powder form) were similar to those of E4, except that the one-pot treatment was conducted for 6 hours, that is, the first mixture was kept at 160 °C and 1.2 MPa for 6 hours.

### Example 6 (E6)

The procedures for preparing the endodontic filling material of E6 (presented in powder form) were similar to those of E4, except that the one-pot treatment was conducted for 10 hours, that is, the first mixture was kept at 160 °C and 1.2 MPa for 10 hours.

### Example 7 (E7)

The procedures for preparing the endodontic filling material of E7 (presented in powder form) were similar to those of E1, except that the one-pot treatment was conducted at 180 °C, that is, the first mixture was kept at 180 °C and 1.2 MPa for 2 hours.

### Example 8 (E8)

The procedures for preparing the endodontic filling material of E8 (presented in powder form) were similar to those of E7, except that the one-pot treatment was conducted for 6 hours, that is, the first mixture was kept at 180 °C and 1.2 MPa for 6 hours.

### Example 9 (E9)

The procedures for preparing the endodontic filling material of E9 (presented in powder form) were similar to those of E7, except that the one-pot treatment was conducted for 10 hours, that is, the first mixture was kept at 180 °C and 1.2 MPa for 10 hours.

### Example 10 (E10)

The procedures for preparing the endodontic filling material of E10 (presented in powder form) were similar to those of E1, except that the one-pot treatment was conducted at 200 °C, that is, the first mixture was kept at 200 °C and 1.2 MPa for 2 hours.

### Example 11 (E11)

The procedures for preparing the endodontic filling material of E11 (presented in powder form) were similar to those of E10, except that the one-pot treatment was conducted for 6 hours, that is, the first mixture was kept at 200 °C and 1.2 MPa for 6 hours.

### Example 12 (E12)

The procedures for preparing the endodontic filling material of E12 (presented in powder form) were similar to those of E10, except that the one-pot treatment was conducted for 10 hours, that is, the first mixture was kept at 200 °C and 1.2 MPa for 10 hours.

### Comparative Example 1 (CE1)

In this example, the tricalcium silicate powder and the tantalum pentoxide powder were mixed in a weight ratio of approximately 5.6:1 using an OptiMix Amalgamator (Kerr) at 25 °C and 1 atm for 30 seconds, thereby obtaining a mixture of the tricalcium silicate powder and the tantalum pentoxide powder of CE1 (hereinafter abbreviated as “mixture of CE1”).

### Comparative Example 2 (CE2)

The procedures for preparing the mixture of the tricalcium silicate powder and the tantalum pentoxide powder of this example were similar to those of CE1, except the tricalcium silicate powder and the tantalum pentoxide powder were mixed in a weight ratio of 4:1. The mixture of the tricalcium silicate powder and the tantalum pentoxide powder of CE2 (hereinafter abbreviated as “mixture of CE2”) was thus obtained.

### <Property Evaluation>

### Determination of Particle Size Distribution

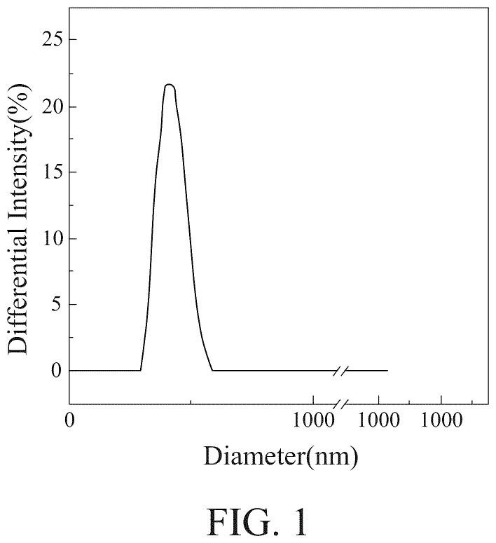

The endodontic filling material of E5 was subjected to determination of particle size distribution using a dynamic light scattering (DLS) particle analyzer (NanoPlus-3), thereby obtaining a DLS graph of the same (see FIG. 1).

Referring to FIG. 1, the endodontic filling material of E5 have particle sizes falling within a range from approximately 300 nm to approximately 600 nm.

### Morphological and Element Analysis

In order to better understand the actual appearance of the above-mentioned endodontic filling materials made by the method according to the present disclosure, the applicant selected the endodontic filling material of E5 for performing the subsequent analysis.

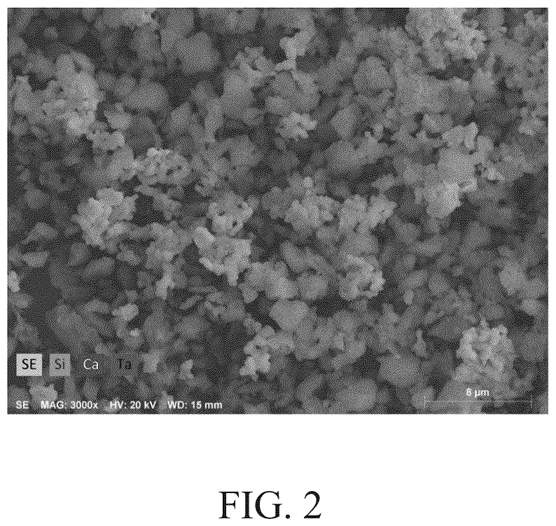

In short, the endodontic filling material of E5 was subjected to observation as well as morphological and element analysis using a scanning electron microscope (SEM) in combination with an energy dispersive X-ray spectroscopy (EDS) system (Hitachi S-4800), thereby obtaining an SEM-EDS image of the same (see FIG. 2).

Referring to FIG. 2, the green color represents silicon atoms, the blue color represents calcium atoms, and the red color represents tantalum atoms; in addition, the foregoing colors may overlap. It can be clearly seen from the SEM-EDS image that particles in the endodontic filling material of E5 indeed can reach a nano-scale size, and the tantalum atoms (i.e., the red dots and pink dots in the SEM-EDS image) are homogeneously distributed in the endodontic filling material of E5, demonstrating successful coating and well distribution of the tantalum pentoxide powder on surfaces of the tricalcium silicate powder.

### X-ray Diffraction (XRD) Analysis

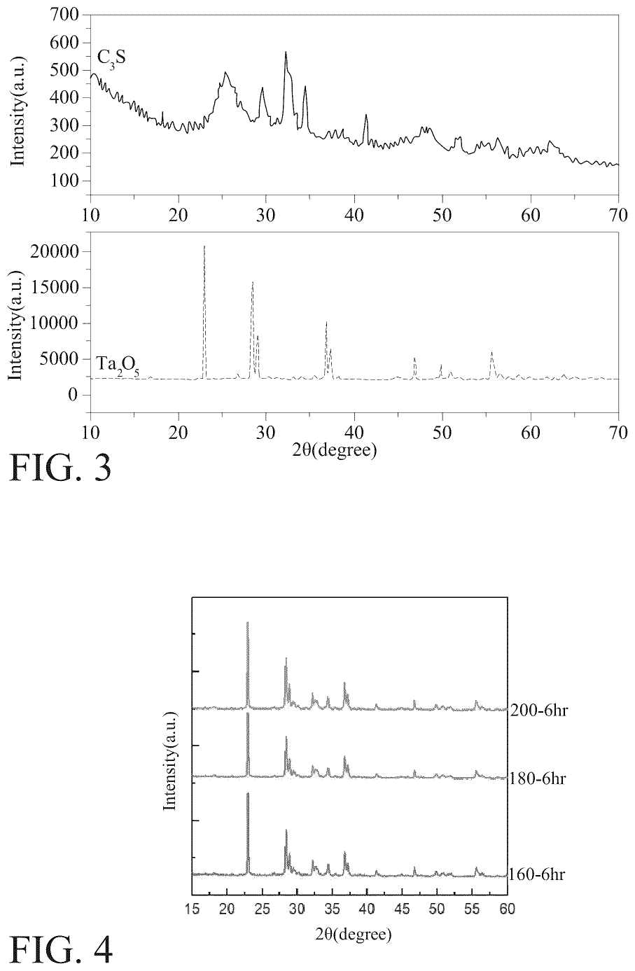

Before conducting the examples mentioned above, each of a suitable amount of the tricalcium silicate powder and a suitable amount of the tantalum pentoxide powder was subjected to XRD analysis using an X-ray diffractometer (Bruker; Model: D2-phaser), thereby obtaining XRD patterns of the same (see FIG. 3). Moreover, the endodontic filling material of each of E5, E8 and E11, was also subjected to XRD analysis using the X-ray diffractometer, thereby obtaining XRD patterns of the same (see FIG. 4). The XRD pattern of each of the tricalcium silicate powder and the tantalum pentoxide powder was provided for comparison.

Referring to FIG. 4, the XRD pattern of the endodontic filling material each of E5, E8 and E11 shows that, after the one-pot treatment adopted in the method according to the present disclosure, the tantalum pentoxide powder can be coated and well distributed on surfaces of the tricalcium silicate powder. Moreover, since the characteristic peaks of the tricalcium silicate powder can be detected in the endodontic filling material of each of E5, E8 and E11, it can be concluded that structure of the tricalcium silicate powder is not altered after the one-pot treatment.

### Application Example

In order to examine the clinical application prospects of the above-mentioned endodontic filling materials, the applicant chose endodontic filling material of each of E1 to E12, and CE1 and CE2 for conducting a radiopacity test as follows.

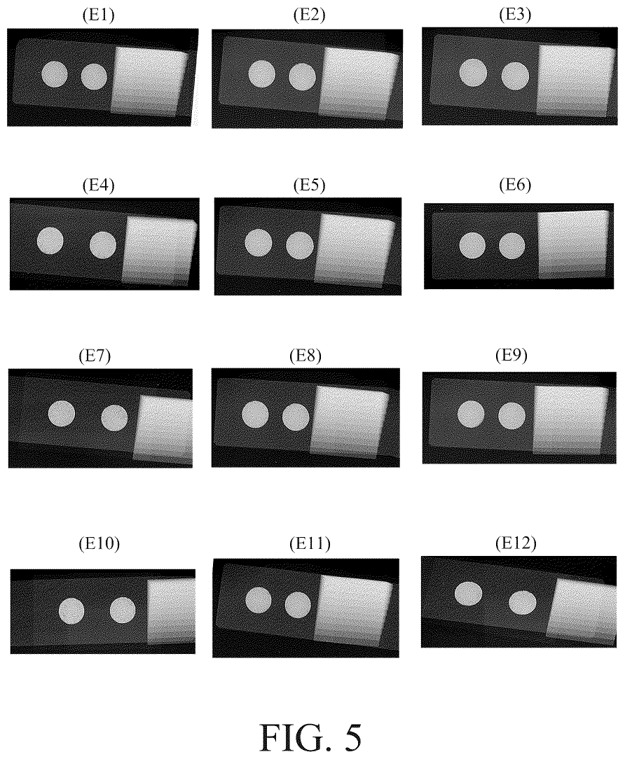

At first, the endodontic filling material of each of E1 to E12, and CE1 and CE2 was mixed with an appropriate amount of distilled water in a weight ratio of 2:1, so as to obtain a sample of each of E1 to E12, and CE1 and CE2, correspondingly. Next, a glass substrate with two smooth depressions, each of which was in a circular shape, on a surface thereof was provided. Thereafter, the sample of each of E1 to E12 was applied on the two smooth depressions. In addition, an aluminum ladder piece with a gradient thickness was disposed on one side of the glass substrate for comparison. Subsequently, the glass substrate having the foregoing sample applied on the two smooth depressions was subjected to exposure of X-ray radiation, thereby obtaining an X-ray image (see FIG. 5).

Referring to FIG. 5, it can be seen from the X-ray image of the sample of each of E1 to E12 that the sample thereof exhibits a good quality in preventing an X-ray from passing therethrough, leading to the endodontic filling material of each of E1 to E12 having sufficient and uniform developing capability, and demonstrating that a good radiopacity effect can indeed be attained by the endodontic filling material of each of E1 to E12. Moreover, from the X-ray image of the sample of each of E2 and E3, E5 and E6, E8 and E9, and E11 and E12, it can be observed that the sample thereof exhibits a better quality in performance of radiopacity effect, showing that having much time for mixing the tricalcium silicate powder and the tantalum pentoxide powder optimizes the distribution uniformity of the tantalum pentoxide powder on the tricalcium silicate powder, thereby enhancing the effect of such

### Sample in Blocking Penetration of X-ray.

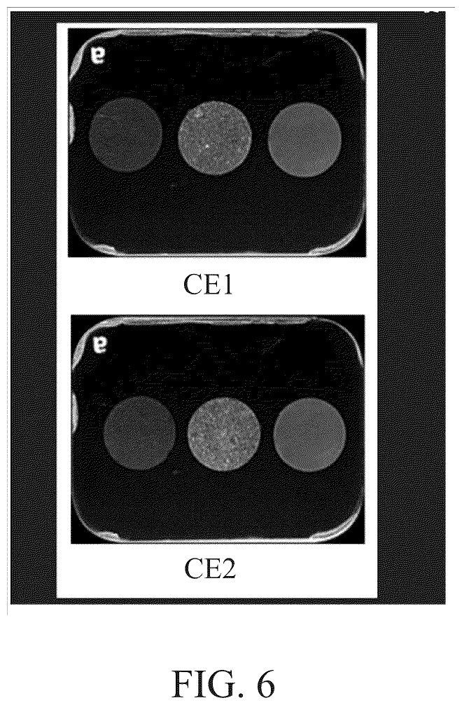

In addition, the sample of each of CE1 and CE2 was also subjected to exposure of X-ray radiation, thereby obtaining an X-ray image of the same (see the two middle circles shown in FIG. 6). For comparison purpose, a tricalcium silicate powder that was mixed with an appropriate amount of distilled water in a weight ratio of 2:1, and Biodentine® (Septodont, Inc.) that was prepared according to the manufacturer's instructions were subjected to exposure of X-ray radiation, thereby obtaining X-ray images of the same (see the circle on the left of each of the two middle circles and the circle on the right of each of the two middle circles, respectively).

Referring to FIG. 6, the samples of both CE1 and CE2 show a bad quality in preventing an X-ray from passing therethrough, which results from uneven distribution and uneven mixing of the tricalcium silicate powder and the tantalum pentoxide powder, causing the X-ray images thereof to appear as an uneven dotted pattern.

In summary, by virtue of the method according to the present disclosure, the endodontic filling material thus prepared possesses nano-scale particle sizes, meaning that particles thereof are extremely fine, and size range of the particles is concentrated (not widely distributed), allowing the endodontic filling material to have good flow stability and enabling it to require less time for setting. Moreover, in such endodontic filling material, the tantalum pentoxide is well distributed and coated on surfaces of the tricalcium silicate, proving an uniform distribution and successful coating of the tantalum pentoxide on the tricalcium silicate is indeed achieved, leading to high uniformity of the endodontic filling material. These results ensure that the endodontic filling material prepared by the method according to the present disclosure is less likely to have phase separation. In addition, although a certain temperature (e.g., 180 °C) and a certain pressure (e.g., 1.2 MPa) are needed in the one-pot solvothermal reaction of the method, no change in structure of the tricalcium silicate is observed after coating of the tantalum pentoxide on the tricalcium silicate, which allows the endodontic filling material to have an improved stability in application. The endodontic filling material is also demonstrated to have high-quality radiopacity due to the presence of the tantalum pentoxide.

Furthermore, since in the method according to the present disclosure, only two principle materials (i.e., the tricalcium silicate and the tantalum pentoxide) involve, and since the procedures of the method are relatively streamlined owing to the one-pot solvothermal reaction, the cost and time in manufacturing of the endodontic filling material can be significantly reduced.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for preparing an endodontic filling material, comprising the steps of:
a) dispersing tricalcium silicate and tantalum pentoxide in a dispersing agent so as to obtain a first mixture;
b) introducing the first mixture into an autoclave, and sealing and heating the autoclave, so that the first mixture is subjected to a one-pot treatment to obtain a second mixture which includes the dispersing agent and an endodontic filling material containing the tricalcium silicate and the tantalum pentoxide coated onto the tricalcium silicate; and
c) subjecting the second mixture to filtration, so as to remove the dispersing agent therefrom, thereby obtaining the endodontic filling material.

2. The method as claimed in claim 1, wherein, in step (a), a weight ratio of the tricalcium silicate to the tantalum pentoxide ranges from 1.5:1 to 3:1.

3. The method as claimed in claim 2, wherein the weight ratio of the tricalcium silicate to the tantalum pentoxide is 3:1.

4. The method as claimed in claim 1, wherein the dispersing agent is ethanol.

5. The method as claimed in claim 1, wherein step a) is carried out by stirring.

6. The method as claimed in claim 5, wherein the stirring is conducted at room temperature for 1 hour.

7. The method as claimed in claim 1, wherein in step b), the one-pot treatment is conducted at a temperature ranging from 120 °C to 220 °C and a pressure ranging from 1 MPa to 1.2 MPa.

8. The method as claimed in claim 7, wherein the temperature ranges from 140 °C to 200 °C

9. The method as claimed in claim 8, wherein the temperature ranges from 160 °C to 200 °C

10. The method as claimed in claim 1, wherein the one-pot treatment is conducted for time period ranging from 2 hours to 10 hours.

11. The method as claimed in claim 10, wherein the time period ranges from 6 hours to 10 hours.

12. The method as claimed in claim 1, wherein the autoclave is heated using an oven.

13. The method as claimed in claim 1, wherein in step c), the filtration is conducted for a time period ranging from 40 seconds to 90 seconds.

14. The method as claimed in claim 1, further comprising, after step c), drying the endodontic filling material at 80 °C for 24 hours.

15. The method as claimed in claim 1, wherein the tricalcium silicate has a particle size ranging from 300 nm to 700 nm.

16. The method as claimed in claim 1, wherein the tantalum pentoxide has a particle size ranging from 300 nm to 800 nm.

17. The method as claimed in claim 1, wherein the endodontic filling material has a particle size ranging from 200 nm to 1000 nm.

18. The method as claimed in claim 17, wherein the particle size ranges from 300 nm to 600 nm.

19. The method as claimed in claim 18, wherein the particle size ranges from 300 nm to 500 nm.
